(19) United States
(12) Patent Application Publication
(10) Pub. No.: US 2025/0090743 A1
(43) Pub. Date: Mar. 20, 2025

(54) CANISTER CONNECTION SYSTEM FOR DISPOSABLE NEGATIVE PRESSURE WOUND THERAPY SYSTEM

(71) Applicant: DeRoyal Industries, Inc, Powell, TN (US)

(72) Inventors: Nicholas J. Poker, Knoxville, TN (US); Ethan Valentine, Knoxville, TN (US); Dhanvin S. Desai, Knoxville, TN (US); Scott Douglas Barnes, Knoxville, TN (US)

(73) Assignee: DeRoyal Industries, Inc, Powell, TN (US)

(21) Appl. No.: 18/368,277

(22) Filed: Sep. 14, 2023

(51) Int. Cl. A61M 1/00

(52) U.S. Cl. CPC A61M 1/98; A61M 1/962; A61M 1/964

(57) ABSTRACT

A disposable NPWT canister connection system having a canister and a canister adapter. The canister includes a pump connection surface having a canister outlet. The canister adapter is operable to fluidly connect the canister to a canister connection surface of a pump unit. The canister adapter including a first pump engagement mechanism positioned and configured to removably engage the first canister engagement mechanism of the canister connection surface, a second pump engagement mechanism positioned and configured to removably engage the second canister engagement mechanism of the canister connection surface, and an aperture disposed between the first pump engagement mechanism and the second pump engagement mechanism. When the first pump engagement and second pump engagement mechanism of the canister are engaging the corresponding first canister engagement mechanism and second canister engagement mechanism, the canister is fluidly connected to the pump unit.

### FIELD

This disclosure relates to the field of negative pressure wound therapy. More particularly, this disclosure relates to a canister connection system particularly suited for disposable negative pressure would therapy systems.

### BACKGROUND

The primary purpose of negative pressure wound therapy (also referred to as “reduced pressure therapy” or “vacuum therapy” in the medical community) is to apply a reduced pressure (also referred to as “negative pressure” or “vacuum pressure” but will be referred to herein as “reduced pressure”) to a wound site to stimulate healing and remove excess exudate from the wound site. The major components of a negative pressure wound therapy system (hereinafter “NPWT system”) include a reduced pressure source (e.g., vacuum pump), a waste canister for collecting extracted exudate from the wound site, a wound enclosure for covering the wound site (e.g., an assembly for directing air flow at the wound site and enclosing various layers such as a drape, dressing foam, etc. as known in the art), and one or more lumens (e.g., tubing) fluidly connecting the wound enclosure to the reduced pressure source and the canister.

Up to the present, NPWT systems have largely been used in hospital settings. This results in longer hospital stays and increased costs to the patient. However, single patient disposable NPWT systems are becoming increasingly available. Disposable systems are generally lightweight and mobile type units that can be worn by the patient for outpatient treatment.

Disposable NPWT systems are typically packaged as a kit. The kit includes a pump unit (which may be reusable), one or more canisters for connection to the pump unit, preassembled dressings that are able to be placed over a wound site, and tubing for fluidly connecting the dressings to the canister. Issues with current disposable systems include providing for a removable fluid tight connection between the pump unit and the canister in a user-friendly and ergonomic manner. Further, many disposable NPWT systems are bulky or otherwise incorporate a canister and associated canister connection system that requires a large footprint. Yet another design consideration is that the connection system should provide the ability to easily monitor the canister fill-status during use.

What is needed, therefore, is an improved canister and associated canister connection system for a disposable NPWT system that solves the foregoing problems.

### SUMMARY

The above and other needs are met by a disposable NPWT system for providing reduced pressure along a fluid flow path to a wound site. The disposable NPWT system includes a pump unit having a canister connecting surface and a canister having a pump connection surface. The canister connecting surface includes a first canister engagement mechanism, a second canister engagement mechanism, and a pump inlet disposed between the first canister engagement mechanism and the second canister engagement mechanism. The canister is configured to be fluidly connected to the pump unit such that the pump connection surface of the canister is positioned against the canister connecting surface of the pump unit. The canister includes a first pump engagement mechanism positioned and configured to removably engage the first canister engagement mechanism, a second pump engagement mechanism positioned and configured to removably engage the second canister engagement mechanism, and a canister outlet disposed between the first pump engagement mechanism and the second pump engagement mechanism. When the first pump engagement and second pump engagement mechanism of the canister are engaging the corresponding first canister engagement mechanism and second canister engagement mechanism, the canister outlet is fluidly sealed to the pump inlet such that the canister is fluidly connected to the pump unit.

According to certain embodiments, the canister includes a canister adapter having the first pump engagement mechanism, the second pump engagement mechanism, and an aperture disposed between the first pump engagement mechanism and the second pump engagement mechanism. The aperture is positioned and configured to be disposed between the pump inlet and the canister outlet when the canister is fluidly connected to the pump unit.

According to certain embodiments, the first pump engagement mechanism includes a deflectable tab, the first canister engagement mechanism includes a first tab receiver and a locking surface disposed adjacent the first tab receiver, and the deflectable tab of the first pump engagement mechanism is configured to deflect inward as it traverses the locking surface of the first canister engagement mechanism and then deflect outward as the deflectable tab passes the locking surface to fluidly connect the canister to the pump unit. According to some embodiments, the second pump engagement mechanism includes an alignment tab and the second canister engagement mechanism includes a second tab receiver dimensioned and configured to receive the alignment tab when the second pump engagement mechanism engages the second canister engagement mechanism.

According to certain embodiments, the canister connecting surface includes a protrusion disposed around the pump inlet and a groove formed into the canister connecting surface around a base of the protrusion. The canister includes a sealing member configured to be disposed around the canister outlet, the sealing member dimensioned and configured to fit within the groove of the canister connecting surface to form a sealed connection between the pump inlet and the canister outlet when the canister is fluidly connected to the pump unit.

According to certain embodiments, the canister further includes a tubing adapter for fluidly connecting to a proximal end of reduced pressure tubing. In some embodiments, the canister connecting surface includes a first recess and a second recess. The first recess includes the pump inlet, first canister engagement mechanism, and the second canister engagement mechanism and is dimensioned and configured to receive the first and second pump engagement mechanisms of the canister. The second recess is dimensioned and configured to receive the tubing adapter.

According to certain embodiments, the disposable NPWT system further includes a door operatively connected to the pump unit, the door for permitting removable engagement of the first and second pump engagement mechanisms with the corresponding first and second canister engagement mechanisms when the door is in an open position, and the door for preventing disengagement of the first and second pump connection engagement mechanisms with the corresponding first and second canister connection engagement mechanisms when the door is in a closed position and the canister is fluidly connected to the pump unit. In some embodiments, the door includes one or more windows for viewing an exudate level of the canister when the door is in the closed position and the canister is fluidly connected to the pump unit.

According to certain embodiments, the disposable NPWT system further includes a wound enclosure and reduced pressure tubing, the reduced pressure tubing configured to be fluidly connected to the canister at a proximal end and to the wound enclosure at a distal end for fluidly connecting the wound enclosure to the canister. According to some embodiments, the wound enclosure includes a filtered vent port for introducing filtered atmospheric air into the reduced pressure tubing for facilitating a flow of exudate from a wound site to the canister through the reduced pressure tubing. According to some embodiments, the proximal end of the reduced pressure tubing is fluidly connected to an inlet port of the canister in a permanent connection and the distal end of the reduced pressure tubing is configured to be fluidly connected to the wound enclosure with a removable connection.

According to another embodiment of the disclosure, a disposable NPWT canister connection system includes a canister having a pump connection surface having a canister outlet and a canister adapter for fluidly connecting the canister to a canister connection surface of a pump unit. The canister connection surface includes a first canister engagement mechanism, a second canister engagement mechanism, and a pump inlet disposed between the first canister engagement mechanism and the second canister engagement mechanism. The canister adapter includes a first pump engagement mechanism positioned and configured to removably engage the first canister engagement mechanism of the canister connection surface, a second pump engagement mechanism positioned and configured to removably engage the second canister engagement mechanism of the canister connection surface, and an aperture disposed between the first pump engagement mechanism and the second pump engagement mechanism. When the first pump engagement and second pump engagement mechanism of the canister are engaging the corresponding first canister engagement mechanism and second canister engagement mechanism to fluidly connect the canister to the pump unit, the canister outlet is fluidly sealed to the pump inlet with the aperture of the canister adapter positioned between the pump inlet and the canister outlet.

According to certain embodiments, the canister adapter is formed of a rigid plastic and the canister is formed of a soft plastic. In some embodiments, the first pump engagement mechanism includes a deflectable tab configured to deflect inward as it traverses a locking surface of the first canister engagement mechanism and then deflect outward as the deflectable tab passes the locking surface to fluidly connect the canister to the pump unit. In some embodiments, the second pump engagement mechanism includes an alignment tab dimensioned and configured to be positioned in a tab receiver of the second canister engagement mechanism to align the canister adapter with the canister connection surface when the second pump engagement mechanism engages the second canister engagement mechanism.

According to certain embodiments, the canister further includes a canister inlet, and the connection system further includes a wound enclosure and reduced pressure tubing. The reduced pressure tubing is configured to be fluidly connected to the canister inlet at a proximal end and to the wound enclosure at a distal end for fluidly connecting the wound enclosure to the canister. In some embodiments, the wound enclosure includes a filtered vent port for introducing filtered atmospheric air into the reduced pressure tubing for facilitating a flow of exudate from a wound site to the canister through the reduced pressure tubing. In some embodiments, the proximal end of the reduced pressure tubing is fluidly connected to the canister inlet in a permanent connection and the distal end of the reduced pressure tubing is configured to be fluidly connected to the wound enclosure with a removable connection. According to some embodiments, the disposable NPWT canister connection system further includes a tubing adapter, the tubing adapter including a bottom surface sealed around the canister inlet and including an inlet port for providing the permanent connection to the proximal end of the reduced pressure tubing.

### BRIEF DESCRIPTION OF THE DRAWINGS

Other embodiments of the invention will become apparent by reference to the Detailed Description in conjunction with the figures, wherein elements are not necessarily to scale so as to more clearly show the details, wherein like reference numbers indicate like elements throughout the several views, and wherein:

FIG. 1 depicts a disposable NPWT system with a front view of the pump unit having a canister fluidly connected to the pump unit according to one embodiment of the disclosure.

FIG. 2 depicts the disposable NPWT system of FIG. 1 with a rear view of the pump unit and fluidly connected canister according to one embodiment of the disclosure.

FIG. 3 depicts a canister connecting surface of the pump unit according to one embodiment of the disclosure.

FIG. 4 depicts a pump connecting surface of a canister according to one embodiment of the disclosure.

FIG. 5 depicts a top portion of the pump connecting surface of the canister as shown in FIG. 4 with a canister adapter secured thereto according to one embodiment of the disclosure.

FIG. 6 depicts the canister adapter and associated canister of FIG. 5 in the process of being connected to a pump unit according to one embodiment of the disclosure.

FIG. 7 depicts the canister adapter and associated canister of FIGS. 5-6 as connected to the pump unit according to one embodiment of the disclosure.

FIG. 8 depicts an alternate embodiment of the canister adapter according to one embodiment of the disclosure.

FIG. 9 depicts a wound enclosure being fluidly connected to a distal end of reduced pressure tubing according to one embodiment of the disclosure.

### DETAILED DESCRIPTION

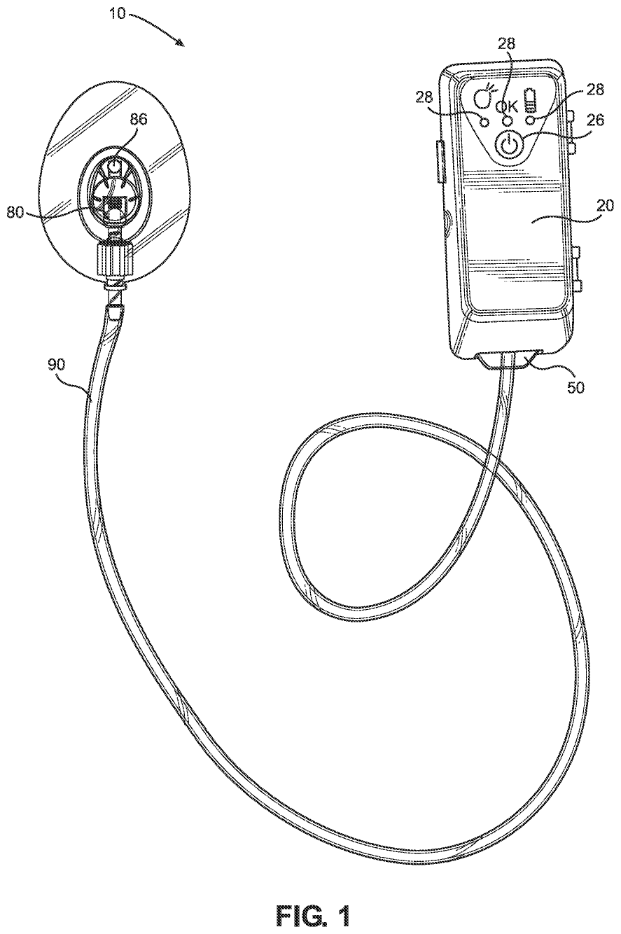

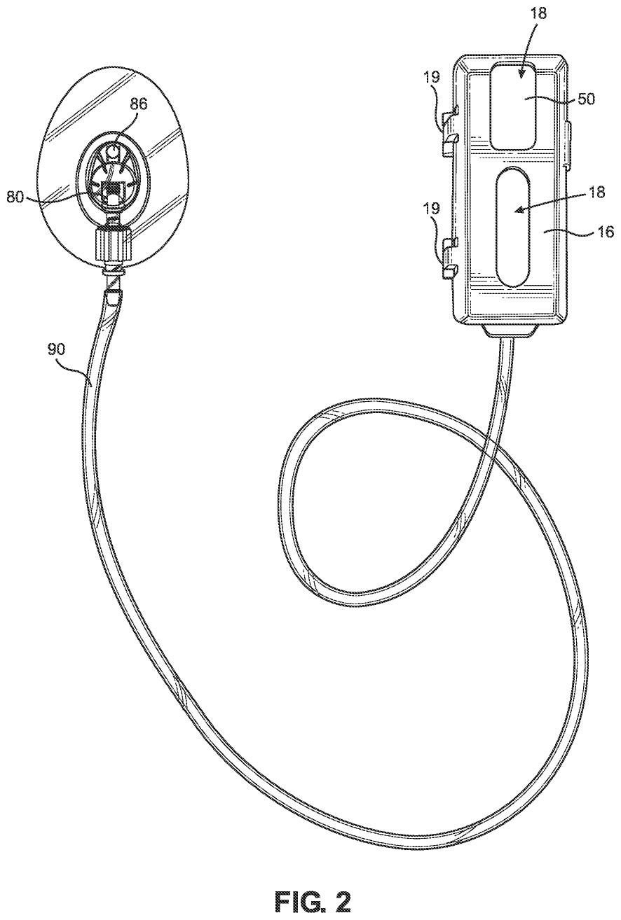

Referring to FIGS. 1-2, an exemplary disposable NPWT system 10 according to the present disclosure is depicted. The system 10 generally includes a pump unit 20, a canister 50 configured to be fluidly connected to the pump unit 20 in a removable manner, a wound enclosure 80 used for placement of a dressing kit over a wound site, and tubing 90 for fluidly connecting the canister 50 to the wound enclosure 80. In operation, the pump unit 20 is operable to provide reduced pressure to the wound enclosure 80 along a reduced pressure flow path such that fluid flows from the wound site to the canister 50 through tubing 90. The reduced pressure generated by the pump unit 20 serves to stimulate healing by applying the reduced pressure to the wound site and removing exudate from the wound enclosure 80 to the canister 50 as known in the art.

According to certain embodiments, a secondary pressure source (e.g., filtered air) may also be provided to provide a secondary pressure to the wound enclosure 80 along a secondary pressure fluid flow path. The secondary pressure flow path is typically fluidly connected to the reduced pressure flow path via the wound enclosure 80 such that fluid from the secondary pressure flow path is pushed into the reduced pressure flow path to form a converged fluid flow path when both the reduced pressure and secondary pressure are delivered to the wound enclosure 80. The converged fluid flow path may be used to facilitate flow of the exudate from the wound site to the canister 50 through the tubing 90. A pressure sensor may also be included along the secondary pressure flow path to measure pressure at the wound site. In embodiments in which the source of the secondary pressure is part of or otherwise positioned within the pump unit 20, the tubing 90 may include dual lumen tubing or separate reduced pressure and secondary pressure tubes for delivering both the reduced pressure and the secondary pressure to the wound site. However, to reduce complexity of the disposable NPWT system 10, and as depicted herein, pump unit 10 is preferably only used as a source of reduced pressure such that tubing 90 is a single lumen tube that only needs to provide reduced pressure to the wound enclosure 80. In embodiments in which a secondary pressure is desired, the secondary pressure source may be provided downstream of the pump unit 20 such as in a filtered air vent 86 at the wound enclosure 80 as depicted herein and further described below.

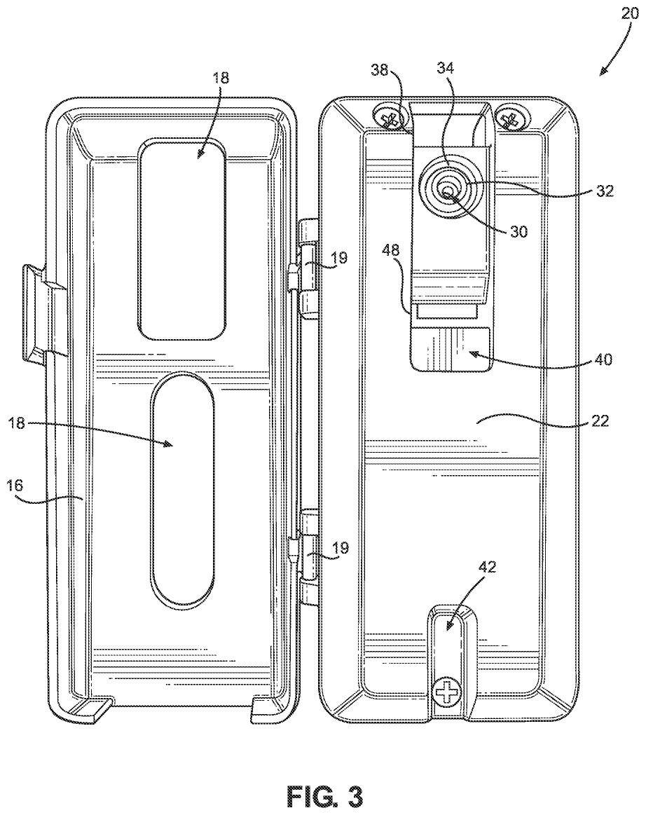

With reference to FIGS. 3-7, the present disclosure is more specifically directed to a disposable NPWT system 10 having an improved canister connection system for removably connecting the canister 50 to the pump unit 20. When the canister 50 is fluidly connected to the pump unit 20, the pump unit 20 is operable to provide reduced pressure through the canister 50 and associated reduced pressure tubing 90 to the wound enclosure 80. In preferred embodiments, the canister 50 is formed of a soft plastic disposable material. A deodorizing agent and/or gelling agent is typically disposed within the canister 50.

With more specific reference to FIG. 3, the pump unit 20 includes a canister connecting surface 22. The canister connecting surface 22 is preferably formed in the rear surface of the unit 20 (with the front surface including the necessary controls 26 such as power and intensity buttons and status indicators/lights 28 as depicted in FIG. 1). However, the canister connecting surface 22 according to the teachings of the present disclosure could also be formed in other surfaces of the unit 20 (e.g., one of the side surfaces).

As shown, the canister connecting surface 22 includes a pump inlet 30. The pump inlet 30 preferably includes a nipple shaped protrusion 32 disposed around the pump inlet 30. A groove 34 is then formed into the canister connecting surface 22 around the base of the protrusion 32. As explained further below, the protrusion 32 and groove 34 help to fluidly connect the pump inlet 30 to the canister outlet 54 of canister 50 in a fluid tight manner.

With continued reference to FIG. 3, the canister connecting surface 22 further includes a first canister engagement mechanism 38 and a second canister engagement mechanism 48. The first and second canister engagement mechanisms 38 and 48 may take various forms but are preferably in the form of tab receivers that are dimensioned and configured to receive corresponding tabs 75 and 77 of the canister 50 as described further below. Disposed between the first and second canister engagement mechanisms 38 and 48 is the pump inlet 30.

In preferred embodiments, and as shown in FIG. 3, the canister connecting surface 22 includes a first recess 40 disposed within a top portion of the canister connecting surface 22 and a second recess 42 disposed within a bottom portion of the canister connecting surface 22. As shown, the first recess 40 contains the pump inlet 30 and first and second engagement mechanisms 38 and 48 described above. The first recess 40 is dimensioned and configured to receive the corresponding engagement mechanisms 74, 76 of the canister adapter 70 described below. The second recess 42 is then dimensioned and configured to receive the tubing adapter 62 described below. Together, the first recess 40 and second recess 42 help provide for a low-profile relationship between a pump connecting surface 52 of the canister 50 and the canister connecting surface 22 of the pump unit 20.

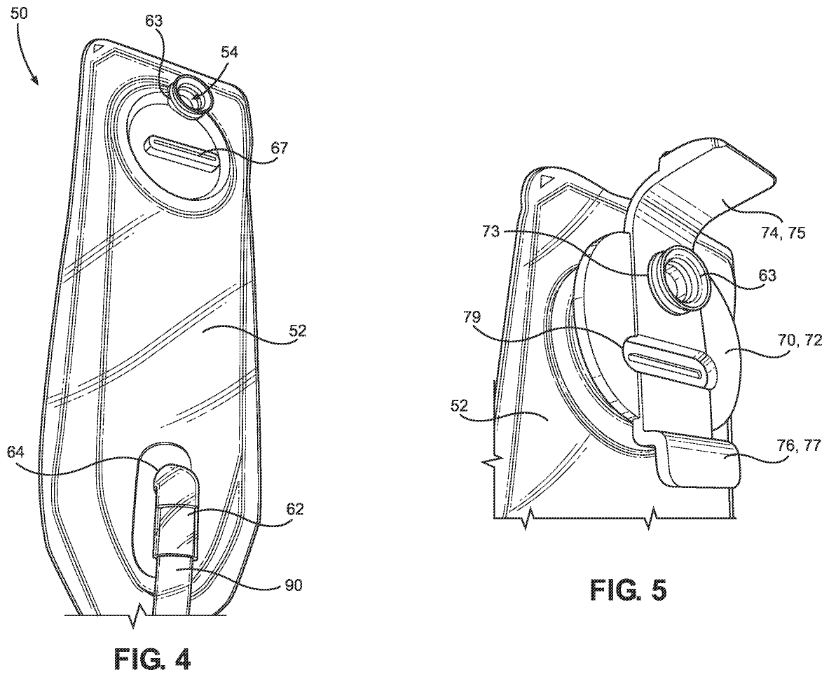

With reference to FIG. 4, the pump connecting surface 52 of canister 50 is depicted according to one embodiment of the disclosure. The pump connecting surface 52 includes a canister outlet 54 that is configured to be fluidly connected to the pump inlet 30 when the canister 50 is connected to the pump unit 20. The canister 50 further includes a canister inlet 64. The canister outlet 54 and canister inlet 64 are fluidly connected via the collection chamber disposed within the canister 50. Reduced pressure tubing 90 is then operable to be fluidly connected at its proximal end to the canister inlet 64 and at its distal end to the wound enclosure 80. In preferred embodiments, the canister 50 and tubing 90 are provided as a pre-packaged unit by sealing the proximal end of the reduced pressure tubing 90 to the canister inlet 64. In more preferred embodiments, the system 10 includes a tubing adapter 62 for fluidly connecting the tubing 90 to the canister inlet 64. For example, a bottom surface of the adapter 64 may be sealed around the canister inlet 64 such that an outlet of the adapter 62 is fluidly connected to the canister inlet 64. The proximal end of the tubing 90 may then be sealed to an inlet of the adapter 62 to fluidly connect the tubing 90 to the canister inlet 64. The adapter 62 is preferably dimensioned and configured to substantially fit within the second recess 42 of the canister connecting surface 22 as described above.

With reference to FIG. 5, the canister connection system further includes a canister adapter 70 configured to be secured to the canister 50 for removably connecting the canister 50 to the canister connecting surface 22 of the pump unit 20. According to this embodiment, the canister adapter 70 includes a base portion 72 having an aperture 73, a first pump engagement mechanism 74, and a second pump engagement mechanism 76. To fluidly connect the canister 50 to the pump unit 20, the canister adapter 70 is positioned such that the first pump engagement mechanism 74 engages the first canister engagement mechanism 38 of the canister connecting surface 22 and the second pump engagement mechanism 76 engages the second canister engagement mechanism 48. When the first pump engagement mechanism 74 and second pump engagement mechanism 76 of the canister 50 are engaging the corresponding first canister engagement mechanism 38 and second canister engagement mechanism 48, the canister outlet 54 is fluidly sealed to the pump inlet 30 through aperture 73 of the canister adapter 70 such that the canister 50 is fluidly connected to the pump unit 20.

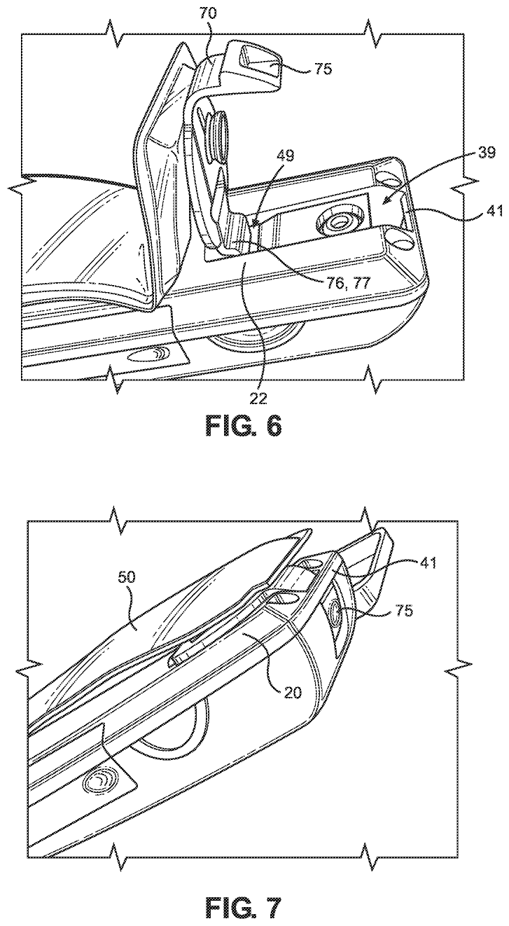

More specifically, the canister adapter 70 is preferably formed of a more rigid piece of plastic as compared to the canister 50. The first pump engagement mechanism 74 and second pump engagement mechanism 76 include respective tabs 75, 77 that extend from opposite ends of the adapter 70. In operation, and referring to FIG. 6, the tab 77 of the second pump engagement mechanism 76 is first inserted into the tab receiver 49 of the second canister engagement mechanism 48. The adapter 70 is then pressed towards the canister connecting surface 22 of the pump unit 20 such that the tab 75 of the first pump engagement mechanism 74 is deflected as it traverses a locking surface 41 of the tab receiver 39 of the first canister engagement mechanism 38. Once the tab 75 traverses the locking surface 41, and as depicted in FIG. 7, the tab 75 of the first pump engagement mechanism 74 returns to its undeflected state to secure the canister 50 to the pump unit 20. To remove the canister 50 from the pump unit 20, the tab 75 is accessible through the top end of the pump unit 20 such that it is operable to be deflected inward to traverse the locking surface 41 in the opposite direction.

While the tabs 75, 77 of canister adapter are shown and described herein with the alignment tab 77 below the deflectable tab 75 (and corresponding receivers 39, 49 of the pump unit 20 positioned accordingly), other positions are possible and within the present disclosure. For example, the engagement mechanisms may be reversed such that alignment tab 77 is disposed above the deflectable tab 75. According to this embodiment, alignment receiver 49 and locking receiver 39 are also reversed accordingly. The tabs 75, 77 and corresponding receivers 39, 49 may also be positioned in a side-by-side configuration. Similarly, the receivers 39, 49 may be positioned anywhere along the canister connecting surface 22 with a corresponding change in positioning being made to the location of the canister adapter 70 with respect to canister 50. Regardless of the chosen positions of the tabs and corresponding receivers, the alignment tab 77 is intended to engage alignment receiver 49 first, and then deflectable tab 75 is inserted into locking receiver 39 second.

As described above with respect to FIG. 3, the canister connecting surface 22 preferably includes a protrusion 32 disposed around the pump inlet 30 and a groove 34 formed around the base of the protrusion 32. With reference to FIGS. 4 and 5, the connection system further includes a sealing member 63 configured to be disposed around the canister outlet 54. The sealing member 63 is dimensioned and configured to fit within the groove 34 around protrusion 32 of the canister connecting surface 22 to help form a fluid tight connection between the pump inlet 30 and canister outlet 54 when the canister 50 is fluidly connected to the pump unit 20. According to certain embodiments, and as shown in FIGS. 4 and 5, the sealing member 63 may be sealed directly to the canister connecting surface 52 of canister 50. According to this embodiment, the aperture 73 of canister adapter 70 is configured to be positioned around the sealing member 63 to secure the canister adapter 70 to the canister 50. In this embodiment, the canister connecting surface 52 of canister 50 may further include a positioning element 67 configured to engage a corresponding positioning element 79 of the canister adapter 70 to ensure and maintain proper positioning of the canister adapter 70 with respect to the canister 50 when the aperture 73 of canister adapter 70 is positioned around sealing member 63. According to this embodiment, the positioning element 67 of the canister 50 is a tab and the positioning element 79 of the canister adapter 70 is a corresponding slot that is dimensioned and configured to receive the tab.

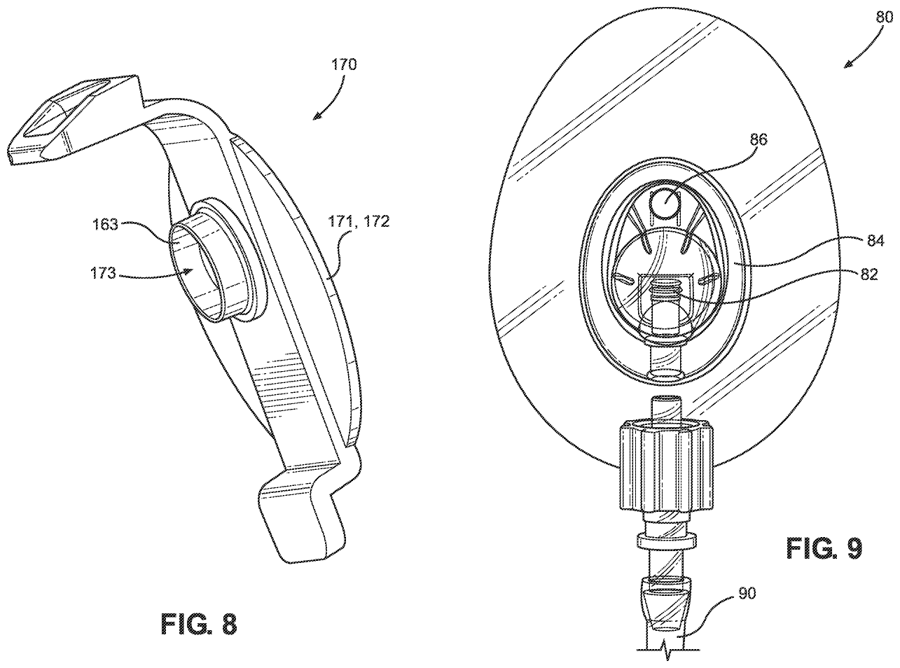

According to an alternate embodiment, and as depicted in FIG. 8, canister adapter 170 may be configured to include a sealing member 163 disposed around aperture 173. Thus, according to this embodiment, the canister 50 includes the canister outlet 54 as described above but sealing member 163 is secured to the canister adapter 170 instead of the canister connecting surface 52. According to this embodiment, rear surface 171 of adapter 170 may be sealed directly to the canister 50 such that the adapter 170 and canister 50 are provided as a single unit (i.e., the canister adapter 170 is intended to be permanently secured to the canister 50).

With reference back to FIG. 3, the disposable NPWT system 10 preferably includes a door 16 operatively connected to the pump unit 20. In operation, the door 16 is intended to substantially cover the canister connecting surface 22 and canister 50 when in a closed position (see FIG. 2). Thus, when in the closed position, the door 16 helps to prevent disengagement of the first and second pump engagement mechanisms 74 and 76 with the corresponding first and second canister engagement mechanisms 38 and 48 by preventing access to or otherwise protecting the canister connection system during use. Similarly, when in the closed position, the door 16 helps to prevent disengagement of the tubing 90 from the canister inlet 64 by covering or otherwise protecting this connection point. On the other hand, when the door 16 is in the open position (see FIG. 3), easy access to the canister connection wall 22 is provided such that the canister 50 may be fluidly connected to the pump inlet 30 in the manner described above and shown in FIGS. 6 and 7.

As shown, the door 16 preferably includes one or more windows 18 for viewing an exudate level of the canister 50 when the door 16 is in the closed position and the canister 50 is fluidly connected to the pump unit 20. Thus, the door 16 is operable to simultaneously prevent disengagement of the canister 50 from the pump unit 20 while still providing the ability of a user to visually track the amount of exudate in the canister 50.

According to certain embodiments, the door 16 is a hinged door that includes one or more hinges 19 for operatively moving the door 16 between the open and closed positions. However, other opening and closing mechanisms are contemplated, such as providing tracks on the sides of the pump unit 20 such that the door is a sliding door that is operable to move up and down with respect to the pump unit 20 for moving the door 16 between the open and closed position.

With reference to FIG. 9, the wound enclosure 80 according to this embodiment includes a dome connector 82 for fluidly connecting the wound enclosure to the distal end of the reduced pressure tubing 90. According to this embodiment, the dome connector 82 is connected to the tubing 90 using a luer lock type connector. The dome connector is then positioned over a flange 84 having a filtered vent 86 disposed in the flange 84. In operation, the wound enclosure 80 is placed over the dressing materials applied to the wound site. The distal end of tubing 90 is then fluidly connected to the dome connector 80 and the canister 50 is fluidly to the pump unit 20. When the pump unit 20 is activated, reduced pressure is provided to the wound site through tubing 90 and dome connector 82. To facilitate the flow of exudate from the wound site to the canister 50 as a result of the reduced pressure delivered by the pump unit, filtered vent 86 provides for filtered atmospheric air to serve as the secondary source of pressure that converges through the wound site and/or enclosure 80 with the reduced pressure of tubing 90.

The foregoing description of preferred embodiments for this disclosure have been presented for purposes of illustration and description. They are not intended to be exhaustive or to limit the disclosure to the precise form disclosed. Obvious modifications or variations are possible in light of the above teachings. The embodiments are chosen and described in an effort to provide the best illustrations of the principles of the disclosure and its practical application, and to thereby enable one of ordinary skill in the art to utilize the disclosure in various embodiments and with various modifications as are suited to the particular use contemplated. All such modifications and variations are within the scope of the disclosure as determined by the appended claims when interpreted in accordance with the breadth to which they are fairly, legally, and equitably entitled.

What is claimed is:

1. A disposable NPWT system for providing reduced pressure along a fluid flow path to a wound site, the disposable NPWT system comprising:
a pump unit having a canister connecting surface, the canister connecting surface including:
a first canister engagement mechanism,
a second canister engagement mechanism, and
a pump inlet disposed between the first canister engagement mechanism and the second canister engagement mechanism,
a canister having a pump connection surface, the canister configured to be fluidly connected to the pump unit such that the pump connection surface of the canister is positioned against the canister connecting surface of the pump unit, the canister including:
a first pump engagement mechanism positioned and configured to removably engage the first canister engagement mechanism,
a second pump engagement mechanism positioned and configured to removably engage the second canister engagement mechanism, and
a canister outlet disposed between the first pump engagement mechanism and the second pump engagement mechanism,
wherein, when the first pump engagement and second pump engagement mechanism of the canister are engaging the corresponding first canister engagement mechanism and second canister engagement mechanism, the canister outlet is fluidly sealed to the pump inlet such that the canister is fluidly connected to the pump unit.

2. The disposable NPWT system of claim 1 wherein the canister includes a canister adapter having the first pump engagement mechanism, the second pump engagement mechanism, and an aperture disposed between the first pump engagement mechanism and the second pump engagement mechanism, the aperture positioned and configured to be disposed between the pump inlet and the canister outlet when the canister is fluidly connected to the pump unit.

3. The disposable NPWT system of claim 1 wherein:
the first pump engagement mechanism includes a deflectable tab,
the first canister engagement mechanism includes a first tab receiver and a locking surface disposed adjacent the first tab receiver, and
the deflectable tab of the first pump engagement mechanism is configured to deflect inward as it traverses the locking surface of the first canister engagement mechanism and then deflect outward as the deflectable tab passes the locking surface to fluidly connect the canister to the pump unit.

4. The disposable NPWT system of claim 3 wherein the second pump engagement mechanism includes an alignment tab, and the second canister engagement mechanism includes a second tab receiver dimensioned and configured to receive the alignment tab when the second pump engagement mechanism engages the second canister engagement mechanism.

5. The disposable NPWT system of claim 1 wherein:
the canister connecting surface includes a protrusion disposed around the pump inlet and a groove formed into the canister connecting surface around a base of the protrusion, and
the canister includes a sealing member configured to be disposed around the canister outlet, the sealing member dimensioned and configured to fit within the groove of the canister connecting surface to form a sealed connection between the pump inlet and the canister outlet when the canister is fluidly connected to the pump unit.

6. The disposable NPWT system of claim 1 wherein the canister further includes a tubing adapter for fluidly connecting to a proximal end of reduced pressure tubing.

7. The disposable NPWT system of claim 6 wherein the canister connecting surface includes:
a first recess including the pump inlet, first canister engagement mechanism, and the second canister engagement mechanism, the first recess dimensioned and configured to receive the first and second pump engagement mechanisms of the canister, and
a second recess dimensioned and configured to receive the tubing adapter.

8. The disposable NPWT system of claim 1 further comprising a door operatively connected to the pump unit, the door for permitting removable engagement of the first and second pump engagement mechanisms with the corresponding first and second canister engagement mechanisms when the door is in an open position, and the door for preventing disengagement of the first and second pump connection engagement mechanisms with the corresponding first and second canister connection engagement mechanisms when the door is in a closed position and the canister is fluidly connected to the pump unit.

9. The disposable NPWT system of claim 8 wherein the door includes one or more windows for viewing an exudate level of the canister when the door is in the closed position and the canister is fluidly connected to the pump unit.

10. The disposable NPWT system of claim 1 further comprising a wound enclosure and reduced pressure tubing, the reduced pressure tubing configured to be fluidly connected to the canister at a proximal end and to the wound enclosure at a distal end for fluidly connecting the wound enclosure to the canister.

11. The disposable NPWT system of claim 10 wherein the wound enclosure includes a filtered vent port for introducing filtered atmospheric air into the reduced pressure tubing for facilitating a flow of exudate from a wound site to the canister through the reduced pressure tubing.

12. The disposable NPWT system of claim 10 wherein the proximal end of the reduced pressure tubing is fluidly connected to an inlet port of the canister in a permanent connection and the distal end of the reduced pressure tubing is configured to be fluidly connected to the wound enclosure with a removable connection.

13. A disposable NPWT canister connection system, the canister connection system comprising:
a canister having a pump connection surface, the pump connection surface including a canister outlet; and
a canister adapter for fluidly connecting the canister to a canister connection surface of a pump unit, the canister connection surface having a first canister engagement mechanism, a second canister engagement mechanism, and a pump inlet disposed between the first canister engagement mechanism and the second canister engagement mechanism, the canister adapter including:
a first pump engagement mechanism positioned and configured to removably engage the first canister engagement mechanism of the canister connection surface,
a second pump engagement mechanism positioned and configured to removably engage the second canister engagement mechanism of the canister connection surface, and
an aperture disposed between the first pump engagement mechanism and the second pump engagement mechanism,
wherein, when the first pump engagement and second pump engagement mechanism of the canister are engaging the corresponding first canister engagement mechanism and second canister engagement mechanism to fluidly connect the canister to the pump unit, the canister outlet is fluidly sealed to the pump inlet with the aperture of the canister adapter positioned between the pump inlet and the canister outlet.

14. The disposable NPWT canister connection system of claim 13 wherein the canister adapter is formed of a rigid plastic and the canister is formed of a soft plastic.

15. The disposable NPWT canister connection system of claim 14 wherein the first pump engagement mechanism includes a deflectable tab configured to deflect inward as it traverses a locking surface of the first canister engagement mechanism and then deflect outward as the deflectable tab passes the locking surface to fluidly connect the canister to the pump unit.

16. The disposable NPWT canister connection system of claim 15 wherein the second pump engagement mechanism includes an alignment tab dimensioned and configured to be positioned in a tab receiver of the second canister engagement mechanism to align the canister adapter with the canister connection surface when the second pump engagement mechanism engages the second canister engagement mechanism.

17. The disposable NPWT canister connection system of claim 13 wherein the canister further includes a canister inlet, the connection system further comprising a wound enclosure and reduced pressure tubing, the reduced pressure tubing configured to be fluidly connected to the canister inlet at a proximal end and to the wound enclosure at a distal end for fluidly connecting the wound enclosure to the canister.

18. The disposable NPWT canister connection system of claim 17 wherein the wound enclosure includes a filtered vent port for introducing filtered atmospheric air into the reduced pressure tubing for facilitating a flow of exudate from a wound site to the canister through the reduced pressure tubing.

19. The disposable NPWT canister connection system of claim 17 wherein the proximal end of the reduced pressure tubing is fluidly connected to the canister inlet in a permanent connection and the distal end of the reduced pressure tubing is configured to be fluidly connected to the wound enclosure with a removable connection.

20. The disposable NPWT canister connection system of claim 19 further comprising a tubing adapter, the tubing adapter including a bottom surface sealed around the canister inlet and including an inlet port for providing the permanent connection to the proximal end of the reduced pressure tubing.
